(19) United States
(12) Patent Application Publication
(10) Pub. No.: US 2010/0198625 A1
(43) Pub. Date: Aug. 5, 2010

(54) METHOD OF DELIVERING AT LEAST ONE PHARMACEUTICAL TO A CLIENT

(76) Inventor: Michael Francis O'Neill, London (GB)

Correspondence Address: QUINN LAW GROUP, PLLC, 39555 ORCHARD HILL PLACE, SUITE # 520, NOVI, MI, 48375, US

(21) Appl. No.: 12/364,846

(22) Filed: Feb. 3, 2009

(51) Int. Cl. G06Q 50/00; G06Q 10/00

(52) U.S. Cl. 705/5; 705/3

(57) ABSTRACT

A method of delivering at least one pharmaceutical to a client, the method comprising the steps of providing a travel itinerary of the client to an order management system, the itinerary including at least one destination; providing a prescription for the at least one pharmaceutical to the order management system; the order management system matching the travel itinerary and the prescription together and then issuing at least one order to at least one pharmaceutical supplier for delivery of the pharmaceutical to the destination before the arrival of the client for collection by the client.

### TECHNICAL FIELD

The present invention relates to a method of delivering at least one pharmaceutical to a client. More particularly, but not exclusively, the present invention relates to a method of providing a prescription and a travel itinerary including a destination to an order management system which matches the two and then issues an order to at least one pharmaceutical supplier to deliver the pharmaceutical for collection by the client at the destination.

### BACKGROUND OF THE INVENTION

Clients often wish to obtain pharmaceuticals whilst at destinations away from home, for example whilst on a trip abroad. There can be a number of reasons for this. For example the client may lose their pharmaceuticals and need replacements. Alternatively they may not wish to carry all of their pharmaceuticals for the whole trip with them. In addition, it can often be cheaper to purchase the pharmaceuticals at the destination, particularly if the destination is abroad. In such cases the client may also wish to purchase pharmaceuticals for import back to the home country.

A problem with purchasing pharmaceuticals whilst away from home is that there may be no guarantee as to the quality of the pharmaceuticals. Instances of counterfeiting are common. Also, clients may occasionally require less common pharmaceuticals, pharmacies at the destinations would need to carry a wide range of stock, some of which may have a low turnover.

### SUMMARY OF THE INVENTION

The present invention seeks to overcome the drawbacks of the prior art. Accordingly, the present invention provides a method of delivering at least one pharmaceutical to a client, the method comprising the steps of providing a travel itinerary of the client to an order management system, the itinerary including at least one destination; providing a prescription for the at least one pharmaceutical to the order management system; the order management system matching the travel itinerary and the prescription together and then issuing at least one order to at least one pharmaceutical supplier for delivery of the pharmaceutical to the destination before the arrival of the client for collection by the client.

At least one pharmaceutical may be delivered to a client. A travel itinerary is stored in a memory that is accessible by a server of an order management system. The itinerary includes at least one destination. A prescription for the at least one pharmaceutical is stored in the memory. At least one pharmaceutical supplier for at least one destination is stored in the memory. The travel itinerary and the prescription are matched together with the at least one pharmaceutical supplier in the server to provide a matched pharmaceutical supplier. Instructions are transmitted through the order management system to the matched pharmaceutical supplier to deliver the at least one pharmaceutical to the destination before arrival of the client at the destination for collection by the client.

An order management system is configured for arranging the delivery of at least one pharmaceutical to a client at a destination abroad based on an itinerary of the client. The system includes a server, a memory, an agent interface, and a supplier interface. The server is connected to the internet wherein a plurality of destinations and a plurality of pharmaceutical supplier are stored therein. The memory is configured for storing information pertaining to at least one of the at least one pharmaceutical, the itinerary, and the at least one pharmaceutical supplier. The memory is in operative communication with the server. The agent interface is interconnected to the server and configured for transmitting and receiving information pertaining to the at least one pharmaceutical from the server. The supplier interface is interconnected to the server and configured for receiving and transmitting information pertaining to the at least one pharmaceutical for the client.

At least one pharmaceutical may be supplied to a client at a remote location, The travel itinerary of the client is stored in a memory, accessible by an order management system, the itinerary including at least one destination. A prescription is stored in the memory for the at least one pharmaceutical. The at least one pharmaceutical supplier is stored in a memory for at least one destination. At least one of the at least one pharmaceutical, the at least one pharmaceutical supplier, and the at least one destination are retrieved from the memory by the server. The travel itinerary and the prescription are matched together with the at least one pharmaceutical supplier in the server to provide a matched pharmaceutical supplier. The at least one matched pharmaceutical supplier is displayed on the agent interface. Instructions are transmitted through the order management system to the matched pharmaceutical supplier to deliver the at least one pharmaceutical to the destination before arrival of the client at the destination for collection by the client.

The system and method according to the invention has a number of advantages. The pharmaceuticals are sourced from a reputable supplier, typically in a country with high standards of pharmaceutical manufacture, packaging and dispensing. The pharmacy at the destination may only receive the pharmaceutical shortly before the arrival of the client so minimising the amount of stock which needs to be carried by the pharmacy at any one time.

Preferably the travel itinerary comprises a plurality of destinations.

Preferably, the order management system issues a plurality of orders to at least one pharmaceutical supplier for delivery of the at least one pharmaceutical at the plurality of destinations.

The travel itinerary can comprise a start point, the start point being in a different country to at least one destination.

The pharmaceutical supplier can be in a different country to the start point.

The travel itinerary can be a cruise itinerary.

The at least one destination can be an island.

Preferably, the order management system issues an identification code on receipt of the travel itinerary.

The step of providing a prescription for the client to the order management system can comprise providing the identification code along with the prescription to the order management system.

Alternatively the order management system issues an identification code on matching the travel itinerary and prescription together.

Preferably, the client provides credit or debit card details along with the identification code to the order management system for payment.

At least one of the travel itinerary and prescription can be provided to the order management system by an agent on behalf of the client.

The above features and advantages and other features and advantages of the present invention are readily apparent from the following detailed description of the best modes for carrying out the invention when taken in connection with the accompanying drawings.

### BRIEF DESCRIPTION OF THE DRAWINGS

Referring now to the figures, which are exemplary embodiments and wherein like elements are numbered alike:

FIG. 1 shows a schematic view of a server interconnected with an agent interface and a supplier interface via the internet;

FIG. 2 shows, in schematic form, a first embodiment of a method according to the invention;

FIG. 3 shows, in schematic form, a further embodiment of a method according to the invention;

FIG. 4 shows, in schematic form, a further embodiment of a method according to the invention; and

FIG. 5 shows, in schematic form, a further embodiment of a method according to the invention.

### DESCRIPTION OF THE PREFERRED EMBODIMENTS

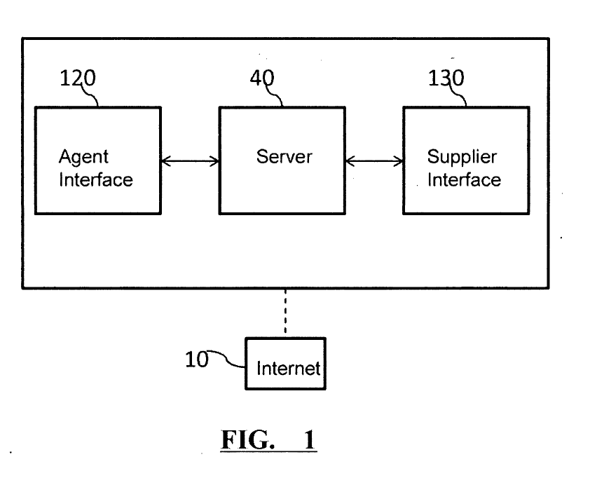

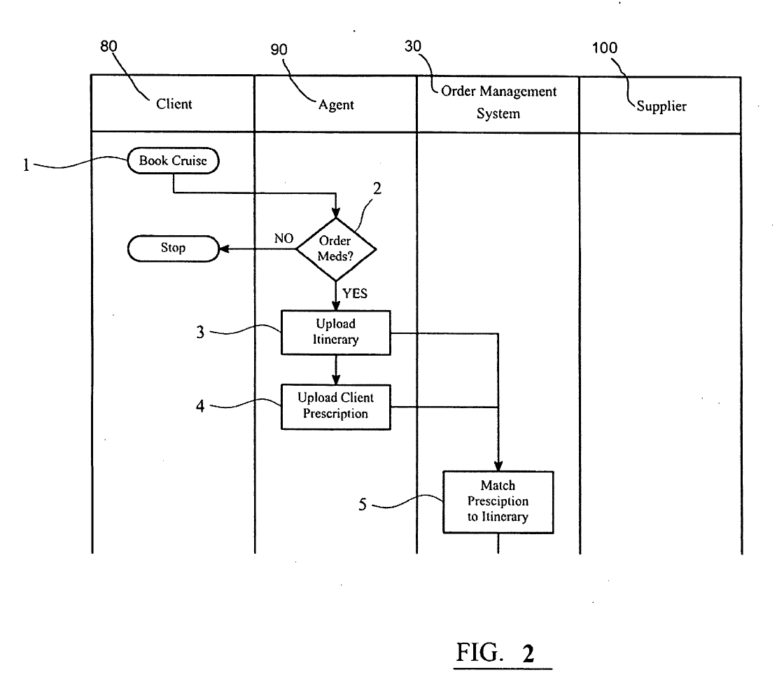

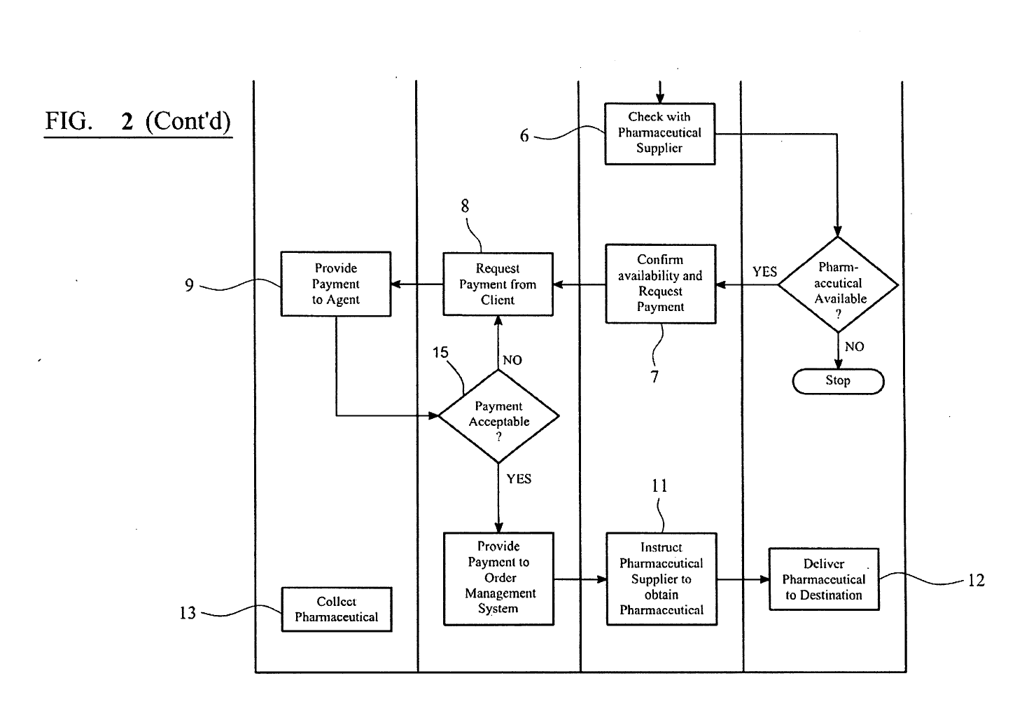

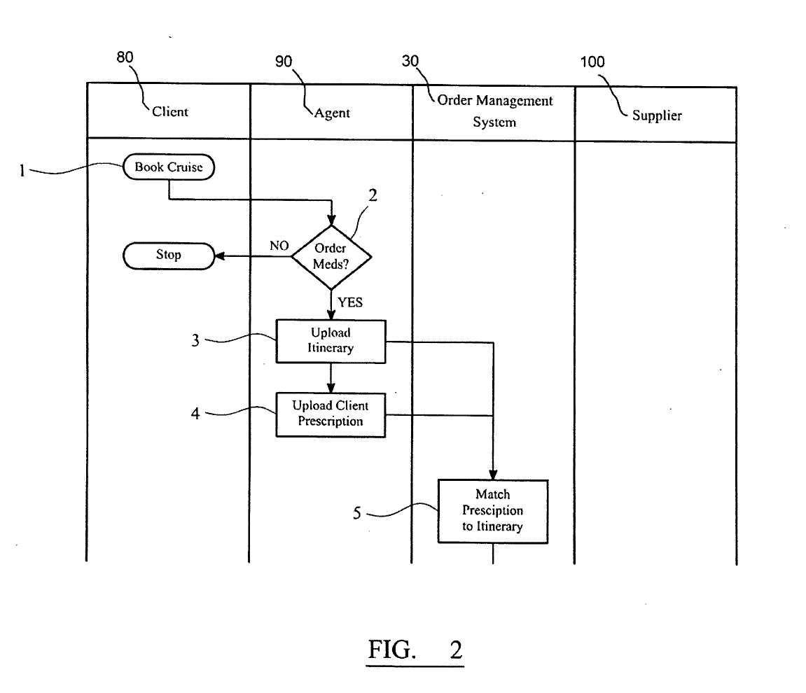

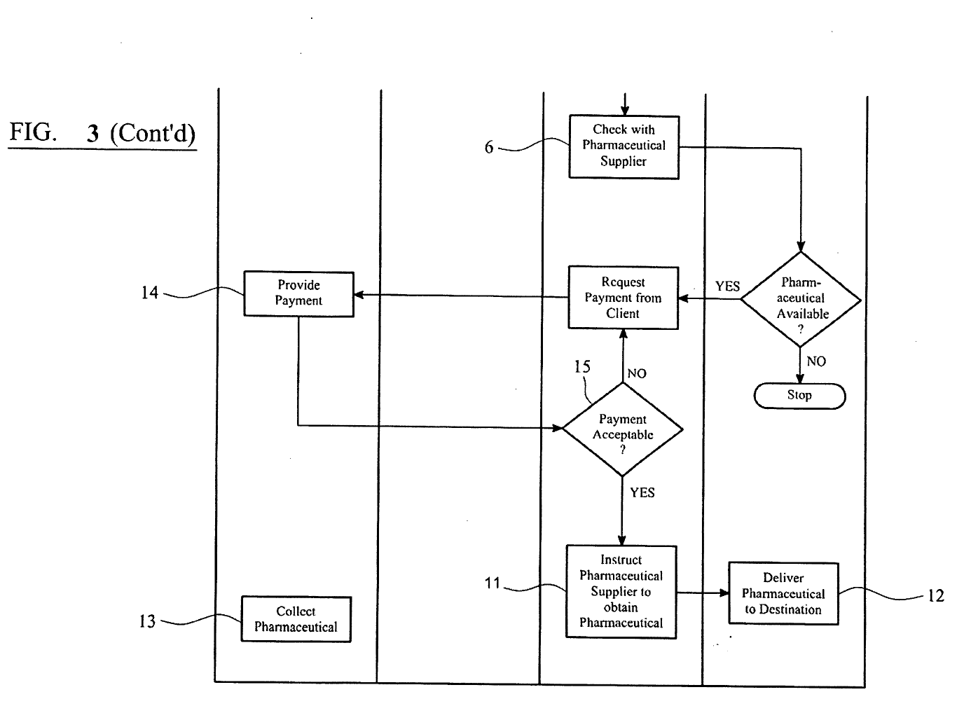

Referring to the drawings, wherein like reference numbers refer to like components, FIG. 1 shows a client management system 30 having a server 40 interconnected with an agent interface 120 and a supplier interface 130 via the internet 110 to arrange the delivery of at least one pharmaceutical to a client 80 at a destination abroad. FIG. 2 shows a first embodiment of a method according to the invention. In a first step a client visits a travel agent 90 and books a trip, in this embodiment a cruise 2, shown at 1 in FIGS. 2 and 3. The cruise starts from a start point and visits a destination abroad, typically an island.

Each destination may include at least one local agent, e.g., a pharmaceutical supplier 100. Once the cruise has been booked, the travel agent 90 offers the client the option of purchasing pharmaceuticals for collection whilst on the cruise, shown as 2 in FIGS. 2 and 3. If the client wishes to purchase such pharmaceuticals the travel agent 90 provides the client's 80 travel itinerary to the order management system 30, shown as 3 in FIGS. 2 and 3. Typically the order management system 30 comprises a server 40 connected to the internet 110. The destinations and the pharmaceutical suppliers 100 are stored on the server 40. The server 40 includes a processor 42 that is configured to process data received via the internet 110 and the like. In addition, the server is in operative communication with at least one memory 44 for storing information and data. The memory 44 may be disposed within the server 40. The memory 44 may also be disposed external to the server 40, i.e., at a remote location, and accessible by the server 40. It should be appreciated that the system 30 may be configured to access one or more memory 44 that are at one or more locations and/or located internal to the server 40. The agent 90 provides the itinerary to the server 40 by means of the agent interface 120, typically a web browser. To do this, the agent 90 stores the travel itinerary of the client in the server 40, where the itinerary includes at least one destination that the client will be visiting.

Once the travel itinerary has been uploaded to the server 40, the agent 90 then uploads the client's 80 prescription to the order management system 30, over the internet 110 via the agent interface 120, such that the prescription is stored in the server 40, shown as 4 in FIGS. 2 and 3. The prescription includes the pharmaceutical to be delivered. The server 40 of the order management system 30 matches the travel itinerary with the prescription 5, shown at 5 in FIGS. 2 and 3, and confirms with at least one pharmaceutical supplier 100 to ensure that the pharmaceutical is available and can be delivered to the required destination 6 by a matched pharmaceutical supplier 100, shown as 6 in FIGS. 2 and 3. The matched pharmaceutical supplier 100 may be displayed on the agent interface for viewing by the agent and/or client. The pharmaceutical supplier 100 views the prescription and the travel itinerary on a supplier interface 130 at the location of the pharmaceutical supplier 100. If the order can be met, i.e., the prescription is available, confirmation is sent to the agent 90 along with a request for payment through the order management system 30, shown as 7 and 8, respectively, in FIGS. 2 and 3. The agent 90 requests payment from the client 80. The client 80 provides payment details to the agent 90, shown as 9 in FIGS. 2 and 3. Assuming the payment details are acceptable, the agent 90 provides payment to the order management system 3.

Once payment has been received, the order management system 30 transmits a request to the matched pharmaceutical supplier 100 instructing the matched pharmaceutical supplier 100 to deliver the pharmaceutical to the destination for collection by the client 80, shown as 11 in FIG. 2. The matched pharmaceutical supplier 100 issues the pharmaceuticals and has them delivered, typically by a third party such as DHL, to the destination, shown as 12 in FIGS. 2 and 3.

When the cruise liner arrives at the destination, the client 80 can collect the pharmaceuticals from a predetermined collection point, typically a pharmacy, shown as 13 in FIGS. 2 and 3. Alternatively, the pharmaceuticals may be delivered to the liner and the like.

The method according to the invention has a number of advantages. Typically the destination is not in the same country as the start of the cruise. The pharmaceutical supplier 100 may also be in a different country to the start of the cruise. The pharmaceuticals can therefore be purchased at a reduced cost compared to the typical cost in the country where the cruise starts. A typical example would be a cruise which starts in the United States and which calls at an island in the Caribbean. Pharmaceuticals are ordered from a pharmaceutical supplier 100 in, for example, Canada and delivered at the Caribbean island. In addition to the cost saving, the client 80 also has a guarantee as to the quality of the pharmaceuticals. The pharmaceuticals arrive in a sealed package so providing a guarantee of quality. As a further advantage, the method according to the invention reduces the stock of pharmaceuticals which must be kept on the destination island. The pharmaceuticals arrive shortly before, or simultaneous with, the client 80.

In the above embodiment the cruise only calls at one destination. In an alternative embodiment the cruise calls at a plurality of destinations where pharmaceuticals may be delivered. When the client 80 visits the agent 90, the client 80 also specifies which of the destinations are to receive deliveries of pharmaceuticals. More than one destination may receive a delivery. Also, there may be more than one type of pharmaceutical delivered either to the same destination or to different destinations. In one embodiment the order management system 30 checks with one or more pharmaceutical suppliers 100 to determine if the client's 80 proposed delivery schedule is possible. If so, the system 30 instructs the agent 90 to take payment as before. If not, the order management system 30 suggests an alternative delivery schedule which fits with the client's 80 travel plans, if one is available.

In an alternative embodiment the agent 90 may provide the prescription and travel itinerary details to an operative of the order management system 30 by phone or by post, rather than over the internet

Shown in FIG. 3 is a further embodiment of the method according to the invention. In this embodiment, rather than the agent 90 taking payment the client 80 provides credit card details direct to the order management system 30, via the agent interface 120 or a similar type of interface. The order management system 30 confirms the credit card details are acceptable, shown at 15 in FIGS. 2 and 3. The order management system 30 then issues the required instructions to the pharmaceutical suppliers 100 for delivery of the pharmaceuticals, shown at 16 in FIG. 3. In an alternative embodiment, once the agent 90 has booked the cruise and provided the client 80 with the travel itinerary the client 80 may provide the prescription, itinerary and payment details direct to the order management system 30, via the agent interface 120 or a similar type of interface.

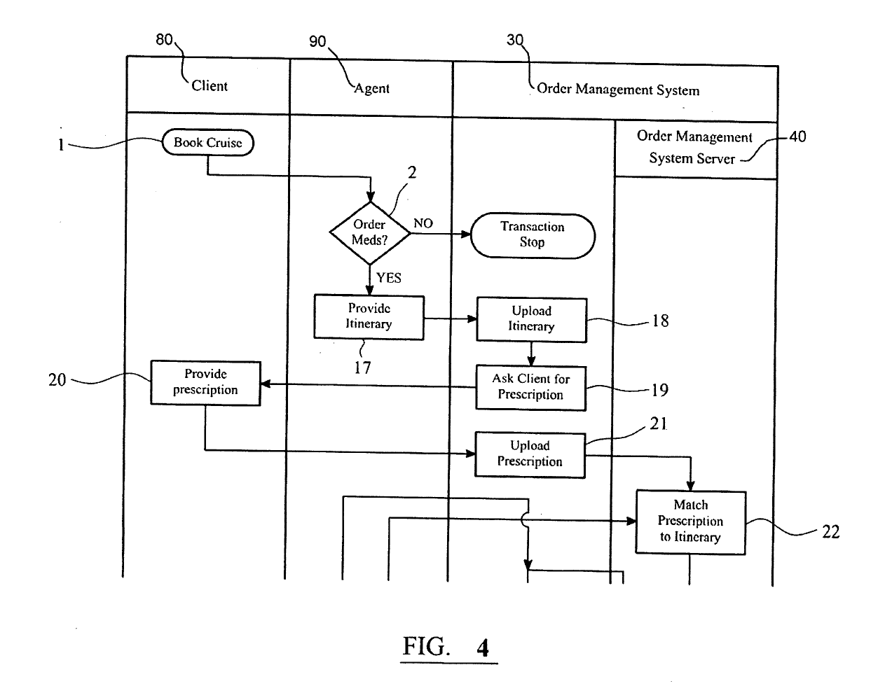

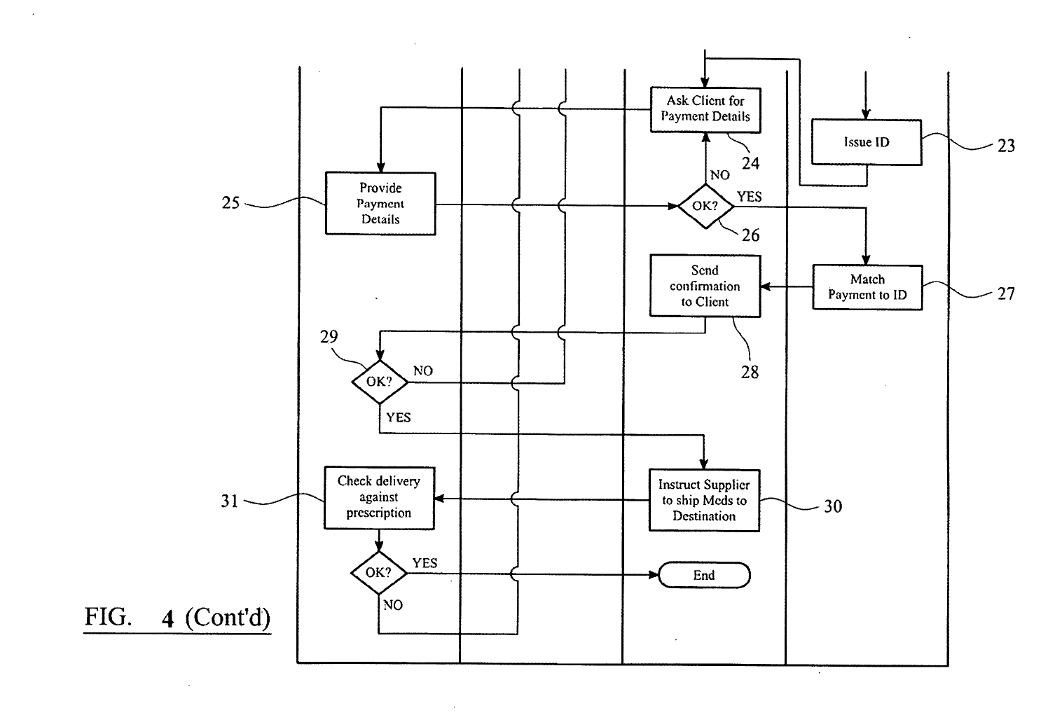

Shown in FIG. 4 is a further embodiment of a method according to the invention. In a first step, shown at 1 in FIG. 3, the client 80 books a cruise through an agent 90. The agent 90 asks the client 80 if they wish to order pharmaceuticals for collection during the cruise 2. If so the agent 90 provides the travel itinerary to the order management system 30 where it is uploaded to the server 40 of the order management system 30, as shown at 30 in FIG. 4. The order management system 30 asks the client 80 for the prescription, as shown at 19 in FIG. 4. The client 80 provides the prescription to the order management system 30, as shown at 20 in FIG. 4, where it is uploaded to the server 40 of the order management system 30. The server 40 matches the prescription to the itinerary, shown at 22 in FIG. 4, and then generates and issues a unique order ID, i.e., an identification code, as shown at 23 in FIG. 4. Therefore, the prescription and the order ID are stored together in the server 40. The order management system 30 then provides the order ID along with a request for payment to the client 80, as shown at 24 in FIG. 4. The client 80 does not need to provide payment at this point. The next time the client 80 logs on to the order management system 30, which may be at a later date, the client 80 uses the order ID to identify the prescription and travel itinerary and to provide payment, as shown at 25 in FIG. 4. The order management system 30 checks the payment details. Assuming the client 80 provides acceptable payment details, as shown at 26 in FIG. 4, the order management system 30 internally matches the payment with the order ID, as shown at 27 in FIG. 4, and transmits a confirmation of receipt of payment to the client 80, as shown at 28 in FIG. 4. The confirmation of receipt of the payment to the client 80 may be displayed on the agent interface 120. The client 80 checks the order confirmation, as shown at 29 in FIG. 4. If details of the order confirmation are not correct the client 80 indicates the appropriate amendments to the order management system 30 which makes the changes and issues a new order ID. The client 80 pays using the new ID and the order management system 30 issues a new confirmation of payment to the client 80. The process is repeated until the client 80 indicates to the order management system 30 they are happy with the order confirmation. The order management system 30 then provides one or more delivery orders to the pharmaceutical suppliers for delivery of the pharmaceuticals at the destination(s), shown at 30 in FIG. 4. The client 80 checks the pharmaceuticals on receipt, shown at 31 in FIG. 4. In an alternative embodiment the client 80 can amend the itinerary and/or prescription before payment.

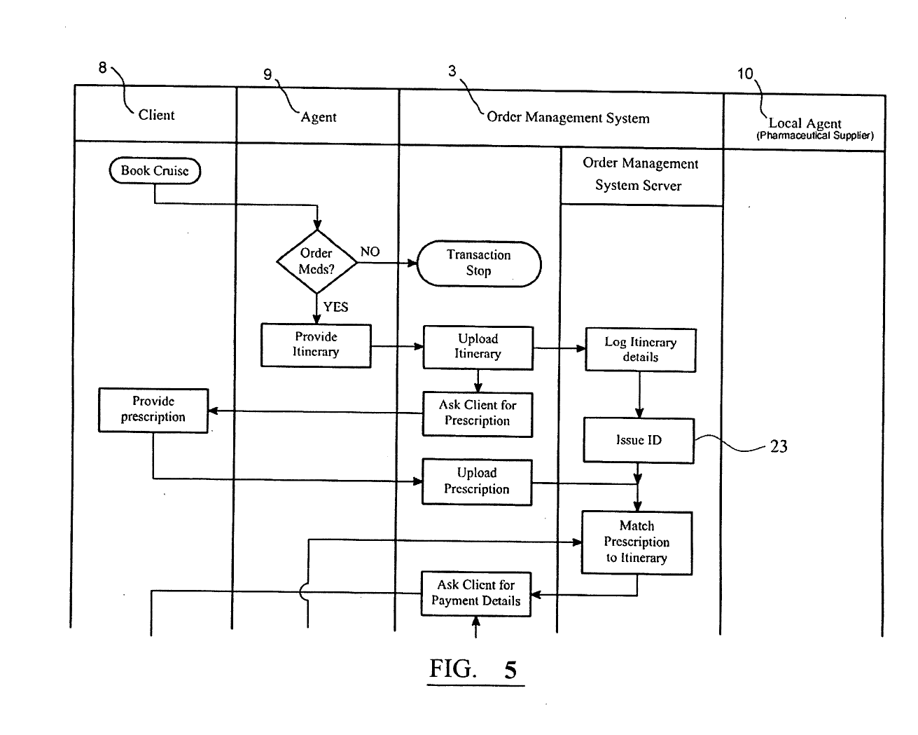

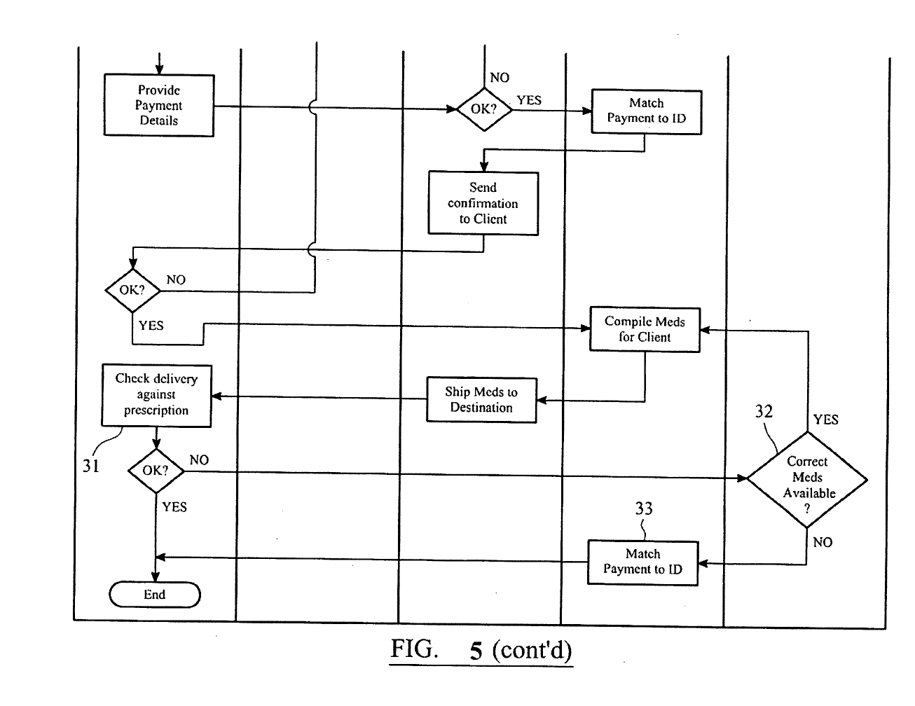

FIG. 5 shows a further embodiment of a method according to the invention. The method of FIG. 5 is similar to that of FIG. 4 except the order management system 30 issues the order ID, as shown at 23 in FIG. 5, after receipt of the travel itinerary but before receipt of the prescription. The client 80 can match the prescription to the travel itinerary at a later date using the order ID. In this embodiment of the method the client 80 checks the pharmaceuticals on receipt, as shown at 31 in FIG. 5. If these are not as expected, the client 80 reports this to the local agent 90 of the order management system 30 at the destination. The local agent 90 checks to see if the pharmaceuticals are available locally, as shown at 32 in FIG. 5, and if so provides them to the client 80. If they are not available locally, the local agent 90 contacts the order management system 30 which orders the pharmaceuticals from a pharmaceutical supplier for delivery at the destination. If this is not possible, the local agent 90 refunds at least a portion of the payment made by the client, as shown at 33 in FIG. 5.

While the best modes for carrying out the invention have been described in detail, those familiar with the art to which this invention relates will recognize various alternative designs and embodiments for practicing the invention within the scope of the appended claims.

1. A method of delivering at least one pharmaceutical to a client, the method comprising the steps of:
storing a travel itinerary of the client in a memory accessible by a server of an order management system, the itinerary including at least one destination;
storing a prescription for the at least one pharmaceutical in the memory;
storing at least one pharmaceutical supplier for at least one destination in the memory;
matching the travel itinerary and the prescription together with the at least one pharmaceutical supplier in the server to provide a matched pharmaceutical supplier;
transmitting instructions through the order management system to the matched pharmaceutical supplier to deliver the at least one pharmaceutical to the destination before arrival of the client at the destination for collection by the client.

2. A method as claimed in claim 1, wherein the travel itinerary comprises a plurality of destinations.

3. A method as claimed in claim 2, further comprising issuing a plurality of orders to at least one pharmaceutical supplier for delivery of the at least one pharmaceutical at the plurality of destinations by the order management system.

4. A method as claimed in claim 1, wherein the travel itinerary comprises a start point, the start point being in a different country to at least one destination.

5. A method as claimed in claim 4, wherein the pharmaceutical supplier is in a different country to the start point.

6. A method as claimed in claim 1, wherein the travel itinerary is a cruise itinerary.

7. A method as claimed in claim 6, wherein the at least one destination is an island.

8. A method as claimed in claim 1, further comprising generating an identification code by the server on receipt of the travel itinerary.

9. A method as claimed in claim 8, wherein the step of storing a prescription for the at least one prescription in the memory is further defined as storing a prescription for the at least one prescription and the identification code in the memory.

10. A method as claimed in claim 1, further comprising issuing an identification code on matching the travel itinerary and prescription together through the management system.

11. A method as claimed in claim 8, further comprising providing at least one of a credit and a debit card detail along with the identification code to the order management system for payment via an agent interface.

12. A method as claimed in claim 1, wherein at least one of the travel itinerary and prescription is provided to the order management system by an agent on behalf of the client via an agent interface.

13. A method as claimed in claim 1, further comprising confirming the availability of the at least one pharmaceutical by the at least one pharmaceutical supplier through the order management system.

14. A method as claimed in claim 13, further comprising requesting payment for the at least one pharmaceutical from the client through the order management system.

15. A method as claimed in claim 14, further comprising providing the payment to the order management system through an agent interface.

15. A method as claimed in claim 15, further comprising displaying a confirmation of receipt of the payment for the at least one pharmaceutical from the client on the supplier interface.

16. A method as claimed in claim 13, wherein confirming the availability of the at least one pharmaceutical is further defined as confirming the at least one pharmaceutical supplier through the order management system on an agent interface.

17. An order management system configured for arranging the delivery of at least one pharmaceutical to a client at a destination abroad based on an itinerary of the client, said system comprising:
a server connected to the internet wherein a plurality of destinations and a plurality of pharmaceutical supplier are stored therein;
a memory configured for storing information pertaining to at least one of the at least one pharmaceutical, the itinerary, and the at least one pharmaceutical supplier;
wherein said memory is in operative communication with said server;
an agent interface interconnected to said server and configured for transmitting and receiving information pertaining to the at least one pharmaceutical from said server; and
a supplier interface interconnected to said server and configured for receiving and transmitting information pertaining to the at least one pharmaceutical for the client.

18. A method of delivering at least one pharmaceutical to a client at a remote location, the method comprising the steps of:
storing a travel itinerary of the client in a memory, accessible by an order management system, the itinerary including at least one destination;
storing a prescription for the at least one pharmaceutical in the memory;
storing at least one pharmaceutical supplier for at least one destination in the memory;
retrieving at least one of the at least one pharmaceutical, the at least one pharmaceutical supplier, and the at least one destination from the memory by the server;
matching the travel itinerary and the prescription together with the at least one pharmaceutical supplier in the server to provide a matched pharmaceutical supplier;
displaying the at least one matched pharmaceutical supplier on the agent interface; and
transmitting instructions through the order management system to the matched pharmaceutical supplier to deliver the at least one pharmaceutical to the destination before arrival of the client at the destination for collection by the client.
